(19) United States
(12) Patent Application Publication
(10) Pub. No.: US 2014/0296594 A1
(43) Pub. Date: Oct. 2, 2014

(54) PROCESS FOR THE FLUID CATALYTIC CRACKING OF OXYGENATED HYDROCARBON COMPOUNDS FROM BIOLOGICAL ORIGIN

(71) Applicant: SHELL OIL COMPANY, Houston, TX (US)

(72) Inventors: Theodorus Johannes BROK, Amsterdam (NL); Yinsuo CAI, Dong Ying (CN); James Lloyd JENKINS, Houston, TX (US); Binghui LI, Sugar Land, TX (US); YiBin LIU, Qingdao (CN); Robert Alexander LUDOLPH, Sugar Land, TX (US); Yunying QI, Sugar Land, TX (US); Colin John SCHAVERIEN, Amsterdam (NL); YongShan TU, Qingdao (CN); ChaoHe YANG, Quingdao (CN)

(73) Assignee: SHELL OIL COMPANY, Houston, TX (US)

(21) Appl. No.: 14/227,767

(22) Filed: Mar. 27, 2014

(30) Foreign Application Priority Data
Mar. 28, 2013 (CN) 201310104798.X

(51) Int. Cl. C07C 7/20; C07C 4/06

(52) U.S. Cl. CPC C07C 7/20; C07C 4/06; C07C 2529/89
USPC 585/310

(57) ABSTRACT

A process for the fluid catalytic cracking of oxygenated hydrocarbon compounds from biological origin. The process comprises (a) contacting a feed comprising the oxygenated hydrocarbon compounds from biological origin with a fluid cracking catalyst at a temperature of equal to or more than 400° C. to produce a products stream; (b) separating fluid cracking catalyst from the products stream and separating a fraction comprising one or more C1-C4 hydrocarbon compounds from the products stream; and (c) processing the fraction comprising one or more C1-C4 hydrocarbon compounds in a work-up process, which comprises one or more oil/water separation steps. One or more de-emulsifiers are added to one or more oil/water separation steps.

### PRIORITY CLAIM

The present non-provisional application claims priority from Chinese application no. 201310104798.X, filed Mar. 28, 2013, the disclosures of which are incorporated herein by reference.

### TECHNICAL FIELD

The present disclosure relates to a process for the fluid catalytic cracking of oxygenated hydrocarbon compounds from biological origin.

### BACKGROUND

Fluid catalytic cracking (FCC) is an important conversion process in present oil refineries. It can be used to convert high-boiling hydrocarbon fractions derived from crude oils into more valuable products such as gasoline components (naphtha), fuel oils and (olefinic) gases (ethene, propene, butene, LPG).

With the diminishing supply of crude petroleum oil, use of renewable energy sources is becoming increasingly important for the production of liquid fuels. These fuels from renewable energy sources are often referred to as biofuels. Such renewable energy sources may also be used as feeds to a fluid catalytic cracking process.

For example, Tian Hua et al. in their article titled “Alternative Processing Technology for Converting Vegetable Oils and Animal Fats to Clean Fuels and Light Olefins”, published in the Chinese Journal of Chemical Engineering, vol. 16 (3), pages 394-400 (2008) describe the fluid catalytic cracking of pure feeds of vegetable oils or animal fats and co-feeds with vacuum gas oil (VGO).

In chapter 7 of Dr. Tian Hua's dissertation titled “Studies on Catalytic Cracking of Fatty Acid Esters”, available from the college of Chemistry and Chemical Engineering, China University of Petroleum (EastChina) since April 2010, Dr. Tian Hua describes that one of the main operation problems experienced when co-processing a 22 wt % bio-feed (a mix of animal and vegetable oil including used cooking oil) with a normal FCC vacuum Gas Oil (VGO) in a commercial Fluid Catalytic Cracking (FCC) unit was severe emulsion formation in the water phase in the main fractionator top accumulator. In the dissertation it is suggested that with the adjustment of operating conditions to an increased riser residence time and an increased riser top temperature (i.e. an increased reaction severity) emulsion formation could be reduced.

From a commercial perspective, however, the suggested increased reaction severity is disadvantageous. Increased temperatures and increased residence times will increase the operating costs of an FCC unit. In addition—on a commercial scale—flexibility in reaction severity may be desired to allow one to change the type of product made to fit market demand.

It would be an advancement in the art to provide a process that may reduce or remove the above described emulsion formation but does not require adjustment of the operating conditions of the FCC reactor(s).

### SUMMARY

Applicant carried out test-runs to establish whether or not part or all of the feed for a commercial FCC unit could be replaced by material of biologic origin, more especially oils and fats of plant or animal origin.

During the test-runs it appeared that when changing the feed in a large (3000 barrels/day) integrated FCC unit from a completely crude mineral oil feed to a feed that comprises a certain amount of oxygenated hydrocarbons from biological origin (in this case more especially 10 wt % of used cooking oil or 10 wt % of tallow oil) immediately problems occurred in water/oil separators located downstream of the FCC reactor. It appeared that emulsions were formed in the oil/water separators rather than the clear separation that was seen when processing only conventional petroleum derived feed. When the addition of feed having a biological origin was stopped, these problems disappeared.

It has now been found that the emulsion formation may be reduced or overcome by the addition of one or more de-emulsifiers to one or more of the oil/water separators. Some embodiments of the present invention therefore provide a process for the fluid catalytic cracking of oxygenated hydrocarbon compounds from biological origin. The process comprises a) contacting a feed comprising the oxygenated hydrocarbon compounds from biological origin with a fluid cracking catalyst at a temperature of equal to or more than 400° C. to produce a products stream; b) separating fluid cracking catalyst from the products stream and separating a fraction comprising one or more C1-C4 hydrocarbon compounds from the products stream; and c) processing the fraction comprising one or more C1-C4 hydrocarbon compounds in a work-up process, which work-up process comprises one or more oil/water separation steps. One or more de-emulsifiers are added to one or more oil/water separation steps. Advantageously, in the above process the formation of emulsions in water/oil separators may be reduced or even completely avoided.

In some embodiments, the one or more oil/water separation steps are carried out in one or more separators chosen from the group consisting of a main fractionator overhead separator, a wet gas compressor discharge separator, one or more high pressure separator(s) and/or a butanizer overhead separator.

In some embodiments, the fraction comprising C1-C4 compounds is cooled to obtain a cooled gas stream and a liquid oil/water condensate, followed by separation of the oil and the water fraction in an oil/water separation step. In some embodiments, the cooled gas stream, before the further separation, is compressed to a pressure between 0.5 and 5 MegaPascal, where after the compressed gas stream is cooled to obtain a cooled compressed gas stream and a liquid oil/water condensate, followed by separation of the oil and the water fraction in an oil/water separation step.

In some embodiments, a fraction comprising C3-C4 compounds is obtained. The fraction is cooled to obtain a cooled gas stream and a liquid oil/water condensate, followed by separation of the oil and the water fraction in an oil/water separation step.

In some embodiments, step c) comprises cooling at least part of the fraction comprising C1-C4 compounds to form a cooled gas/liquid mixture and separating the cooled gas/liquid mixture in a main fractionator overhead separator into a cooled gas stream, an oil phase and a water phase. In some embodiments, the process can further comprise compressing at least part of the cooled gas stream to form a compressed gas/liquid mixture and separating the compressed gas/liquid mixture in a wet gas compressor discharge separator into a compressed gas stream, an oil phase and a water phase. In some embodiments, the process can further comprise washing the compressed gas stream one or more times with water and/or steam to form one or more washed gas/liquid mixture(s) and separating such one or more washed gas/liquid mixture(s) in one or more high pressure separator(s) into a washed compressed gas stream, an oil phase and a water phase. In some embodiments, the process can further comprise separating the, optionally washed, compressed gas stream into a dry gas stream comprising C1-C2 hydrocarbon compounds and a LPG stream comprising C3-C4 hydrocarbon compounds, cooling the LPG stream comprising C3-C4 hydrocarbon compounds to form a cooled LPG gas/liquid mixture and separating the cooled LPG gas/liquid mixture in a butanizer overhead separator into an LPG gas stream, an oil phase and a water phase; wherein one or more de-emulsifiers are added to one or more of the separators.

In some embodiments, the fraction comprising C1-C4 compounds comprises products of catalytically cracking of a tri-glycerides and/or catalytically cracking of one or more free fatty acids. In some embodiments, the one or more de-emulsifiers are added to the streams entering a oil/water separator or to the emulsions in a oil/water separator. In some embodiments, the one or more de-emulsifiers are chosen from the group consisting of (alkyl)phenol-formaldehyde resins, epoxy resins, amines, polyamines, amides, di-epoxides, alcohols, polyols, polyol block copolymers, and the alkoxylated, especially ethoxylated or propoxylated, derivatives there from. In some embodiments, the process according to anyone of the preceding claims, wherein the one or more de-emulsifiers are added in an amount of equal to or less than 0.1 vol %, and equal to or more than 1 ppmv (parts per million by volume) of the total liquid stream going into a separator.

Other advantages and features of embodiments of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DETAILED DESCRIPTION OF PREFERRED EMBODIMENTS

The present invention relates to a process for the fluid catalytic cracking of oxygenated hydrocarbon compounds from biological origin. Such fluid catalytic cracking (FCC) processes can suitably be carried out in fluid catalytic cracking (FCC) units comprising one or more fluid catalytic cracking (FCC) reactors.

Modern FCC units can operate continuous processes that may operate 24 hours a day for a period of two to four years. An extensive description of FCC technology can for example be found in “Fluid Catalytic Cracking technology and operations”, by Joseph W. Wilson, published by PennWell Publishing Company (1997) and “Fluid Catalytic Cracking; Design, Operation, and Troubleshooting of FCC Facilities” by Reza Sadeghbeigi, published by Gulf Publishing Company, Houston Texas (1995).

Step a) of some embodiments of the invention comprises contacting a feed comprising the oxygenated hydrocarbon compounds from biological origin with a fluid cracking catalyst at a temperature of equal to or more than 400° C. to produce a products stream. In this step, the oxygenated hydrocarbon compounds from biological origin and optionally any petroleum, natural gas or coal derived co-feed may be cracked in a fluid catalytic cracking (FCC) process.

By a hydrocarbon compound is herein preferably understood a compound comprising at least one hydrogen and at least one carbon atom bonded to eachother by at least one covalent bond. By an oxygenated hydrocarbon compound is herein preferably understood a hydrocarbon compound further comprising at least one oxygen atom, which oxygen atom is covalently bonded to at least one carbon atom.

The feed used in some embodiments of the invention comprises oxygenated hydrocarbon compounds from a biological origin. Such compounds from a biological origin may herein also be referred to as bio-feeds or biorenewable feedstocks, as opposed to petroleum-derived feeds and petroleum-derived feedstocks. By a biological origin is herein preferably understood that they are derived from a biological source as opposed to for example a petroleum derived source, a natural gas derived source or a coal derived source. Without wishing to be bound by any kind of theory it is believed that such compounds derived from a biological origin may preferably contain carbon-14 isotope in an abundance of about 0.0000000001%, based on total moles of carbon.

The hydrocarbon compounds used as a feed in some embodiments of the invention may at least partially be derived from a biological source, or may be wholly derived from a biological source.

Any oxygenated hydrocarbon compounds from a biological origin may be used in some embodiments of the invention. Examples of suitable oxygenated hydrocarbon compounds include those present in triglycerides, pyrolysis oils, liquefied biomass, solid biomass material and/or mixtures thereof. Examples of feeds comprising oxygenated hydrocarbon compounds from biological origin include triglyceride containing feeds, such as vegetable oils, animal fat and/or used cooking oil. Examples of suitable vegetable oils include palm oil, canola oil, rapeseed oil, coconut oil, corn oil, soya oil, castor oil, cottonseed oil, seaweed oil, safflower oil, sunflower oil, linseed oil, olive oil and peanut oil. Examples of suitable animal oils or fats include pork lard, beef fat, mutton fat and chicken fat, fish oil, yellow and brown greases.

In a preferred embodiment, the feed comprising oxygenated hydrocarbon compounds from biological origin may include a solid biomass material. An advantage of using a solid biomass material in the feed is that it may allow one to simplify processes, as for example operation units for liquefaction of a biomass are not needed. More preferably the solid biomass material is not a material used for food production. Examples of preferred solid biomass materials include aquatic plants and algae, agricultural waste and/or forestry waste and/or paper waste and/or plant material obtained from domestic waste. Such a solid biomass material may contain oxygenated hydrocarbon compounds from biological origin such as cellulose and/or lignocellulose. Examples of suitable cellulose- and/or lignocellulose-containing feeds include agricultural wastes such as corn stover, soybean stover, corn cobs, rice straw, rice hulls, oat hulls, corn fibre, cereal straws such as wheat, barley, rye and oat straw; grasses; forestry products and/or forestry residues such as wood and wood-related materials such as sawdust; waste paper; sugar processing residues such as bagasse and beet pulp; or mixtures thereof. More preferably the solid biomass material is selected from the group consisting of wood, sawdust, straw, grass, bagasse, corn stover and/or mixtures thereof. Such solid biomass materials are advantageous as they do not compete with food production and are therefore considered more sustainable.

In another preferred embodiment, the feed comprises oil and/or fats from plant sources, including algae and seaweed, fish or animal sources or microbial sources. Preferably the oxygenated hydrocarbon compounds from a biological origin are compounds derived from plant oil, animal fat or used cooking oil. Most preferably, the oxygenated hydrocarbon compounds from a biological origin comprise mono-, di- and/or tri-glycerides and/or free fatty acids (FFA's). In a most preferred embodiment, the feed may therefore comprise one or more mono-, di- and/or tri-glycerides and/or one or more free fatty acids (FFA's). Such tri-glycerides and FFA's may for example contain aliphatic hydrocarbon chains in their structure having 9 to 22 carbons.

Plant and animal oils and fats may for example contain 0-30 wt % free fatty acids, which are formed during hydrolysis (e.g. enzymatic hydrolysis) of triglycerides. The amount of free fatty acids present in vegetable oils may for example be 1-5 wt % and in animal fat, 10-25 wt %. Without wishing to be bound by any kind of theory it is further believed that during the FCC step, any mono-, di- or tri-glycerides may be converted into one or more free fatty acids. For example it is believed that during step a) free fatty acids and mono-, di- or tri-glycerides may be converted into one or more C4-C22 free fatty acids, possibly one or more C4-C12 free fatty acids or even C5-C10 free fatty acids.

In a preferred embodiment the feed used in the process according to the invention may for example include tallow or used cooking oil. In another preferred embodiment, the feed in the process according to the invention contains tall oil. Tall oil is a by-product of the wood processing industry. Tall oil may contain rosin esters and rosin acids in addition to FFA's. Rosin acids are cyclic carboxylic acids, rosin esters are the esters thereof. For the process, the feed can include a single oil or a mixture of two or more oils, in any proportions. Triglycerides may be transesterified before use into alkylcarboxylic esters as formiates, acetates etc.

In another preferred embodiment, the feed may contain pyrolysis oil or liquid biocrude. By pyrolysis is herein understood the thermal decomposition of a, preferably solid, cellulosic material at a temperature of equal to or more than 350° C., preferably a temperature in the range from 400° C. to 600° C. Such a pyrolysis process is preferably carried out under oxygen-depleted or oxygen-free circumstance. In an especially preferred embodiment the feed may contain pyrolysis oil may be obtained by so-called flash or fast pyrolysis. Biocrudes may conveniently be obtained by liquefaction (also referred to as solvolysis) or hydroliquefaction of a cellulosic material.

Preferred feeds include liquid biofeeds, especially used cooking oil and tallow oil. The feed in some embodiments of the invention may in addition to the bio-feed comprise a conventional crude oil (also sometimes referred to as a petroleum oil or mineral oil), an unconventional crude oil (that is, oil produced or extracted using techniques other than the traditional oil well method) or a Fisher Tropsch oil (sometimes also referred to as a synthetic oil) and/or a mixture and/or derivates of any of these.

In some embodiments of the present invention, in principle, the whole feed may be a biofeed. Suitably the amount of oxygenated hydrocarbon compounds may be up to 65 vol % of the total feed, preferably between 1 and 45 vol %, more preferably between 2 and 35 vol %, even more preferably between 3 and 25 vol % or even between 4 and 15 vol %. The remaining part of the feed may be a petroleum derived feed.

Petroleum derived feeds for the FCC process, which may also be used together with the bio-feeds in some embodiments of the present invention, are preferably high boiling oil fractions, having an initial boiling point of at least 240° C., or even at least 320° C., suitably at least 360° C. or even at least 380° C. (at a pressure of 0.1 MegaPascal). Examples of suitable petroleum derived co-feeds include straight run (atmospheric) gas oils, vacuum gas oil (VGO), flashed distillate, coker gas oils, or atmospheric residue (‘long residue’) and vacuum residue (‘short residue’). Preferred petroleum derived feeds are VGO or long residue. Most preferably heavy gas oils are used, or (high) vacuum gas oils. In addition, high boiling fractions from other refinery units, e.g. the thermal cracker, the hydrocracker and catalytic dewaxing units, may be used.

The feed of some embodiments of the present invention further may or may not contain a certain amount of sulphur. That is, the feed in some embodiments of the invention may comprise the oxygenated hydrocarbon compounds from biological origin and an amount of sulphur. The sulphur may be present in any petroleum derived part of the feed and/or in the biofeed. In practice, more than 70 wt % on total sulphur, or even more than 90 wt % on total sulphur, may be originating from a petroleum derived co-feed. The sulphur may be present in the form of organic sulphur, e.g. sulphide, disulphides and/or aromatic sulphur compounds. The sulphur content in the feed may preferably be equal to or less than 6 wt % sulphur based on total feed, more preferably equal to or less than 4 wt %, even more preferably equal to or less than 3 wt %, and most preferably between 0.1 and 2.5 wt %, based on total weight of the feed. Due to the reaction conditions during fluid catalytic cracking, the sulphur present in the feed may for a large part be converted into hydrogen sulphide. Further, mercaptans may be produced.

In addition the feed may or may not contain one or more nitrogen-containing compounds. These nitrogen-containing compounds may include one or more basic nitrogen compounds.

Some embodiments of the invention comprise a step wherein the feed comprising the oxygenated hydrocarbon compounds from biological origin is contacted with a fluid cracking catalyst at a temperature of equal to or more than 400° C. to produce a products stream. This step may herein also be referred to as FCC or fluid catalytic cracking step. Such an FCC step may suitably be carried out in a so-called FCC unit, suitably in a FCC reactor. This FCC unit may comprise one or more FCC reactor(s) (preferably so-called riser reactor(s)); one or more regenerators; and one or more separators. The separators may include separators for separating the catalyst and a so-called main fractionator to separate the products stream into several fractions.

For example, preheated feed, preheated preferably to a temperature between 160 and 420° C., more preferably between 180 and 380° C., may be injected into a riser reactor, where it may be vaporized and cracked into smaller molecules by contacting and mixing with hot fluid cracking catalyst from a regenerator. Preferably a recycle stream from the main fractionator is simultaneously injected into the reactor. Also (transport) steam may be injected into the riser reactor. The cracking reactions may take place in the reactor within a period of between 0.3 and 12 seconds, preferably between 0.6 and 5 seconds.

The riser reactor may be an elongated tubular reactor having for example a diameter between 0.2 and 2.5 m, preferably 0.5 to 1.5 meter and a length between 8 and 32 m, preferably between 12 and 24 m. The reaction temperature in the riser reactor is preferably between 400 and 750° C., the pressure is preferably between 0.1 and 0.3 MegaPascal. In a preferred embodiment of the present invention the feed is contacted with the fluid cracking catalyst at a temperature in the range of from equal to or more than 460° C. to equal to or less than 610° C., and the contact time between the feed and the fluid catalytic catalyst is preferably less than 10 seconds, more preferably between 0.5 to 8 seconds.

The catalyst/feed weight ratio is preferably between 4 and 50, more preferably between 5 and 35, even more preferably between 6 and 20. The hydrocarbon vapors and/or transportation steam may fluidize the, preferably powdered, catalyst and the mixture of hydrocarbons and catalyst may flow upwards through the riser reactor to enter a separation unit where a products stream comprising cracked hydrocarbons may be separated from the “spent” fluid cracking catalyst.

Separating fluid cracking catalyst from the products stream may preferably be carried out by one or more horizontal and/or vertical cyclones, often in two or more stages. Preferably at least 96 wt % of the spent fluid cracking catalyst is removed from the products stream comprising cracked hydrocarbons, preferably 98 wt %, more preferably 99 wt %. The spent catalyst particles preferably flow down via a stripping unit in which by means of steam stripping further product hydrocarbons may be removed from the spent catalyst particles. From there the spent catalyst particles can be sent to the regenerator unit. The cracking reactions generally produce an amount of carbonaceous material (often referred to as coke) that usually deposit on the catalyst, which may result in a quick reduction of the catalyst activity. The catalyst can be regenerated by burning off the deposited coke with air blown into the regenerator. The amount of coke can for example be between 2 and 10 wt % based on the feed. Hot flue gas may leave the top of the regenerator through one or more stages of cyclones to remove entrained catalyst from the hot flue gas. The temperature in the regenerator is preferably between 640 and 780° C., the pressure is preferably between 0.15 and 0.35 MegaPascal (MPa). The residence time of the catalyst in the regenerator is preferably between five minutes and 2 hours.

The fluid cracking catalyst can be any catalyst known to the skilled person to be suitable for use in a cracking process. Preferably, the fluid cracking catalyst comprises a zeolite. In addition, the fluid cracking catalyst can contain an amorphous binder compound and/or a filler. Examples of the amorphous binder component include silica, alumina, titania, zirconia and magnesium oxide, or combinations of two or more of them. Examples of fillers include clays (such as kaolin).

The zeolite is preferably a large pore zeolite. By a large pore zeolite is herein preferably understood a zeolite comprising a porous, crystalline aluminosilicate structure having a porous internal cell structure on which the major axis of the pores is in the range of 0.62 nanometer to 0.8 nanometer. The axes of zeolites are depicted in the ‘Atlas of Zeolite Structure Types’, of W. M. Meier, D. H. Olson, and Ch. Baerlocher, Fourth Revised Edition 1996, Elsevier, ISBN 0-444-10015-6. Examples of such large pore zeolites include FAU or faujasite, preferably synthetic faujasite, for example, zeolite Y or X, ultra-stable zeolite Y (USY), Rare Earth zeolite Y (=REY) and Rare Earth USY (REUSY). According to the present invention USY is preferably used as the large pore zeolite.

The fluid cracking catalyst can also comprise a medium pore zeolite. By a medium pore zeolite is herein preferably understood a zeolite comprising a porous, crystalline aluminosilicate structure having a porous internal cell structure on which the major axis of the pores is in the range of 0.45 nanometer to 0.62 nanometer. Examples of such medium pore zeolites are of the MFI structural type, for example, ZSM-5; the MTW type, for example, ZSM-12; the TON structural type, for example, theta one; and the FER structural type, for example, ferrierite. According to the present invention, ZSM-5 is preferably used as the medium pore zeolite.

In some embodiments of the present invention steam may be introduced in the process at a number of positions. Thus, steam may be introduced for instance at the lower end of the riser reactor, half way the riser reactor, in the stripper unit and in the transport pipe of spent catalyst to the regenerator. Steam may for example be added to the feed/fluid cracking catalyst and/or to the stripper unit to improve the separation of the catalyst from the products stream. Further the feed to the FCC process may contain a certain amount of water.

The products stream obtained after the separation of the catalyst, for example at a temperature in the range from 400 to 660° C., preferably between 460 and 610° C., and for example at a pressure in the range from 0.1 to 0.3 MegaPascal (MPa), and optionally the vapors from the stripping unit may flow to the lower section of a fractionator (also referred to herein as main fractionator). This fractionator is preferably a distillation column in which the products stream may be separated into fractions. Suitably at least 60 wt % of the products stream from the fluid catalytic process may be introduced into the main fractionator, more suitably at least 80 wt % and preferably the whole products stream is introduced in the main fractionator. In the main fractionator the products can be separated into FCC end-products. The main products may include for example a fraction comprising one or more C1-C4 hydrocarbon compounds (which may be part of the so-called offgas), naphtha, gasoline, light cycle oil, a heavier fraction suitable as fuel oil (sometimes two fractions are separated, light fuel oil and heavy fuel oil) and a slurry oil. Some FCC units produce a light and a heavy naphtha fraction. The slurry oil is preferably returned to the riser reactor. Also a part or all of one or more of the heavier fractions may be returned to the riser reactor.

In this manner a fraction comprising one or more C1-C4 hydrocarbon compounds can be obtained. By a Cx compound is herein understood a compound containing x carbon atoms. The fraction comprising one or more C1-C4 hydrocarbon compounds may comprise or consist of the above mentioned offgas or a fraction thereof. In a preferred embodiment, the fraction comprising one or more C1-C4 hydrocarbon compounds may also comprise hydrogen, nitrogen, hydrogen sulphide and/or water and/or steam. In addition, without wishing to be bound by any kind of theory, it is believed that the fraction comprising one or more C1-C4 hydrocarbon compounds may also contain one or more free fatty acids. The free fatty acids that may be present in the fraction comprising one or more C1-C4 hydrocarbon compounds may possibly include free fatty acids having in the range from 4 to 22, possibly in the range from 4 to 12, preferably in the range from 5 to 10 carbon atoms. For example, the fraction comprising one or more C1-C4 hydrocarbon compounds may include one or more free fatty acids chosen from the group consisting of butanoic acid, butenoic acid, pentanoic acid, pentenoic acid, hexanoic acid, hexenoic acid, heptanoic acid, heptenoic acid, octanoic acid, octenoic acid, nonanoic acid, nonenoic acid, decanoic acid and decenoic acid.

In an especially preferred embodiment of the invention the catalyst is suitably separated from the products stream and the separated products stream is fractionated in a distillation column into one fraction comprising one or more C1-C4 hydrocarbon compounds and at least one further fraction; whereafter the fraction comprising the one or more C1-C4 hydrocarbon compounds is preferably further separated into a fraction comprising one or more C1-C2 hydrocarbon compounds (also referred to herein as dry gas fraction) and a fraction comprising one or more C3-C4 hydrocarbon compounds (also referred to herein as LPG fraction). In addition to the Cx hydrocarbon compounds, one or both fractions may also contain hydrogen, hydrogen sulphide, water and /or nitrogen. The dry gas fraction (the fraction comprising one or more C1-C2 compounds) may for example include methane, ethane and/or ethene. The LPG fraction (the fraction comprising one or more C3-C4 compounds) may for example include propane, propene, butane and butene.

In a preferred embodiment, the fraction comprising one or more C1-C4 hydrocarbon compounds from the products stream can be obtained by feeding a separated products stream to a distillation column, fractionating the cracked products stream into an offgas fraction comprising C1-C4 compounds and at least one further fraction, optionally followed by separating fraction comprising the C1-C4 fraction into a fraction comprising mainly C1-C2 compounds (i.e. more than 80 mol % based on hydrocarbons) and a fraction comprising mainly C3-C4 compounds (i.e. more than 80 mol % based on hydrocarbons).

In step c) of some embodiments of the invention, the fraction comprising one or more C1-C4 hydrocarbon compounds is processed in a work-up process, which work-up process comprises one or more oil/water separation steps, where one or more de-emulsifiers are added to one or more oil/water separation steps. In a preferred embodiment, step c) comprises separating the fraction comprising C1-C4 hydrocarbon compounds in one or more further fractions in a work-up process, which work-up process comprises one or more oil/water separation steps; wherein one or more de-emulsifiers are added to one or more oil/water separation steps.

During the work-up process mentioned in step c), liquid water and/or steam may be present which originates for example from steam used as a lift-gas in the FCC step or from steam or liquid water formed in-situ during the FCC step; or which originates from water used in one or more washing cycles. The work-up process mentioned in step c) may therefore involve the presence or use of water and/or steam.

In several stages of the work-up process such water and/or steam may need to be separated from one or more hydrocarbon compounds. In a preferred embodiment, the work-up process comprises one or more oil/water separation steps carried out in one or more separators; wherein one or more de-emulsifiers are added to such one or more separators. Such separators may suitably including or consist of one or more oil/water separators. The separators may further include one or more combined gas/oil/water separator(s) and/or one or more separate gas/liquid separator(s) and/or liquid/liquid (oil/water) separator(s). Examples of such separators include for example the main fractionator overhead separator, the wet gas compressor discharge separator, one or more high pressure separator(s) and/or the butanizer overhead separator.

For example a C1-C4 compound containing fraction obtained from the top of the main fractionator (also referred to herein as main fractionator offgas, or main fractionator vapours) may suitably be cooled and partially condensed in one or more coolers. The coolers may include air coolers and/or water-cooled trim condensers. In this manner, a cooled gas/liquid mixture may be obtained that is suitably forwarded to a main fractionator overhead separator. This main fractionator overhead accumulator is sometimes also referred to as Main Fractionator Overhead Drum or Main Fractionator Overhead Accumulator.

When merely processing a conventional petroleum derived feed in the FCC unit, a liquid in this main fractionator overhead separator may conveniently form two phases. The two phases may comprise an oil phase, containing for example the hydrocarbon compounds, and a water phase, suitably containing condensed steam. After formation of such an oil phase and such a water phase, the phases may conveniently be separated by phase separation. In view of the presence of hydrogen sulphide, this water phase may sometimes also be referred to as sour water. Due to the feed of oxygenated hydrocarbon compounds of a biological origin in the FCC unit, however, an emulsion may form in this main fractionator overhead separator. This emulsion can be an unstable emulsion that may settle within for example 1 to 4 hours; or the emulsion can be a stable emulsion that does not settle within for example 4 hours.

In some embodiments of the present invention the formation of an emulsion in the main fractionator overhead separator may be reduced or avoided all together by the addition of one or more de-emulsifiers to the main fractionator overhead separator. The one or more de-emulsifiers may be added separately to the main fractionator overhead separator or they may be added in one or more of the streams leading to the main fractionator overhead separator and be supplied to the main fractionator overhead separator via one or more of these streams.

From the cooled gas/liquid mixture mentioned above, a cooled gas stream may be separated. The cooled gas stream may suitably be forwarded to a gas recovery unit (GRU). This gas recovery unit is sometimes also referred to as gas concentration unit. Part or all of the cooled gas stream (suitably equal to or more than 60 vol %, especially equal to or more than 80 vol %) may be sent to the gas recovery unit, preferably all of the cooled gas stream is sent to the gas recovery unit. In the gas recovery unit the cooled gas stream, suitably obtained from the main fractionator overhead separator, may be compressed in a wet gas compressor. In the wet gas compressor the gas stream is preferably compressed to a pressure between 0.5 and 5 MegaPascal (MPa), preferably between 1.0 to 2.5 MPa. This suitably results, suitably after cooling, in the formation of a compressed gas/liquid mixture. The compressed gas/liquid mixture may suitably be forwarded to a wet gas compressor discharge separator (also sometimes referred to as wet gas compressor discharge drum). Again, when merely processing a conventional petroleum derived feed in the FCC unit, the liquid in this wet gas compressor discharge separator may conveniently form an oil phase and a water phase, and this oil phase and water phase (also referred to as sour water) may conveniently be separated by phase separation. Due to the feed of oxygenated hydrocarbon compounds of a biological origin in the FCC unit, however, also here an emulsion may form in this wet gas compressor discharge separator. This emulsion can be an unstable emulsion that may settle within for example 1 to 4 hours; or the emulsion can be a stable emulsion that does not settle within for example 4 hours. In some embodiments of the present invention, the formation of an emulsion in this wet gas compressor discharge separator may be reduced or avoided all together by the addition of one or more de-emulsifiers to the wet gas compressor discharge separator. The one or more de-emulsifiers can be added directly to the wet gas compressor discharge separator or they can be added via one of the streams feeding into the wet gas compressor discharge separator.

From the compressed gas/liquid mixture mentioned above, a compressed gas stream may be separated. The compressed gas stream may optionally be washed one or more times with water and/or steam to form one or more washed gas/liquid mixtures, whereafter the liquid may be separated in an oil phase and a water phase in one or more high pressure separator(s). The washings may be carried out co-currently, counter-currently or in parallel such as for example explained by Joseph W. Wilson in his handbook titled “Fluid Catalytic Cracking Technologies and Operations”, published 1997 by PennWell Publishing Company, pages 238-241 and especially FIGS. 8.6, 6.7 and 8.8. In the process of the present invention the formation of an emulsion in one or more of such high pressure separator(s) may also be reduced or avoided all together by the addition of one or more de-emulsifiers. The one or more de-emulsifiers may be added to such a high pressure separator directly or via one of the streams thereto, for example via the water wash stream.

It is also possible to add additional water and/or steam to the compressed gas/liquid mixture before forwarding such compressed gas/liquid mixture to the wet gas compressor discharge separator. The, optionally washed, compressed gas stream may subsequently be separated into a so-called dry gas stream (i.e. a gas comprising hydrogen, methane, ethane, ethene and optionally nitrogen) and a so-called LPG stream (i.e. a stream comprising C3-C4 hydrocarbon compounds such as propane, propene, butane and butane). Optionally also saturated and unsaturated compounds may be separated.

The, optionally washed, compressed gas stream is sent to the lower section of an absorber, also referred to as primary absorber. Suitably a hydrocarbon liquid, such as a naphtha fraction or a gasoline fraction of the main fractionator (possibly an unstabilized naphtha fraction, i.e. a naphtha fraction containing C4-minus hydrocarbon compounds), is introduced in the upper section of the primary absorber. From the upper part of the primary absorber, a dry gas stream can be obtained. From the bottom part of the primary absorber, a rich hydrocarbon liquid containing C3-C4 hydrocarbon compounds such as propane, propene, butane and butane may be obtained. The dry gas may optionally be introduced in the lower section of a so-called sponge absorber (also referred to as secondary absorber), as described for example by Joseph W. Wilson in his handbook titled “Fluid Catalytic Cracking Technologies and Operations”, published 1997 by PennWell Publishing Company, pages 246-247. In this secondary absorber the dry gas may be contacted with a so-called sponge oil. In this way it is assured that the dry gas only contains C2 compounds and compounds having a lower molecular weight. The rich sponge oil may be regenerated and the regenerated hydrocarbon liquid may be introduced as feed in the primary absorber. The rich hydrocarbon liquid containing C3-C4 hydrocarbon compounds obtained from the primary absorber is preferably either directly or indirectly (for example via a gas/oil/water separator system) introduced in the upper part of a stripper column. In the stripper column any C1 or C2 compounds, and optionally some C3 compounds, may be removed from the hydrocarbon liquid. The purified hydrocarbon liquid obtained from the stripper column is preferably sent to a debutanizer column, in which C3-C4 hydrocarbon compounds may be separated from the hydrocarbon liquid, for example to produce FCC naphtha product (also referred to as stabilized FCC naphtha). Conveniently a liquid C3-C4 hydrocarbon compound stream is obtained from the debutanizer column as a light, gaseous top fraction. After cooling, this light fraction may yield a gas/liquid mixture, which cooled gas/liquid mixture may be sent to a so-called butanizer overhead separator (also sometimes referred to as butanizer overhead drum). In the process of the present invention the formation of an emulsion in this butanizer overhead separator may also be reduced or avoided all together by the addition of one or more de-emulsifiers. The one or more de-emulsifiers may be added to such a butanizer overhead separator directly or via one of the streams thereto.

As explained above, in accordance with some embodiments of the invention, one or more de-emulsifiers may be added to one or more of the above mentioned separators, such as for example a main fractionator overhead separator, a wet gas compressor discharge separator, one or more high pressure separator(s) and/or a butanizer overhead separator. Each of such separators independently may comprise a combined gas/oil/water separator or may comprise a combination of a gas/liquid separator and a liquid/liquid (oil/water) separator.

In accordance with some embodiments of the invention, the one or more de-emulsifiers may therefore be added to one or more oil/water separation steps, wherein such one or more oil/water separation step may be carried out in one or more separators chosen from the group consisting of a main fractionator overhead separator, a wet gas compressor discharge separator, one or more high pressure separator(s) and/or a butanizer overhead separator.

Without wishing to be bound by any kind of theory, it is believed that the formation of the emulsions may be due to the presence of products from catalytically cracking triglycerides and/or catalytically cracking of free fatty acids. It is believed that even ppmv (parts per million by volume) of free fatty acids themselves may contribute to the formation of emulsions. The products from catalytically cracking triglycerides and/or catalytically cracking of free fatty acids and/or triglycerides may include free fatty acids which may be present in the fraction comprising one or more C1-C4 hydrocarbon compounds, and which may be carried over to any oil/water separation steps in any oil/water separators.

Such free fatty acids may include free fatty acids having in the range from 4 to 22, possibly in the range from 4 to 12, preferably in the range from 5 to 10 carbon atoms, for example butanoic acid, butenoic acid, pentanoic acid, pentenoic acid, hexanoic acid, hexenoic acid, heptanoic acid, heptenoic acid, octanoic acid, octenoic acid, nonanoic acid, nonenoic acid, decanoic acid and decenoic acid. The free fatty acids may be considered to have a hydrophobic head and a hydrophilic tail and therefore may possibly act as surfactant or enhance surfactant behaviour.

Again, without wishing to be bound by any kind of theory, it is believed that due to the bio-feed in the FCC step, the concentration of such oxygen containing C1-C4 hydrocarbon compounds (such as the above free fatty acids) in the above mentioned separators respectively the above mentioned separation steps may have increased compared to a conventional FCC feed and such increased concentration may lead to the emulsion formation.

According to some embodiments of the invention, one or more de-emulsifiers can be added to the streams entering the oil/water separator and/or to the emulsions in the oil/water separator. The de-emulsifiers are herein also referred to as de-emulsifying agents. In principle every compound that breaks emulsions can be used. Commercially available de-emulsifiers may be used. Such demulsifying agents are often intended to break emulsions of crude oil fractions and water, but may also be used in the specific application of the present invention. Preferably the one or more de-emulsifiers are chosen from the group consisting of (alkyl)phenol-formaldehyde resins, epoxy resins, amines, polyamines, amides, di-epoxides, alcohols, polyols, polyol block copolymers, and the alkoxylated, especially ethoxylated or propoxylated and/or derivatives there from. Commercially available de-emulsifiers may comprise a mixture of two to four different de-emulsifying agents in a carrier solvent (e.g. xylene, (heavy) naphtha, isopropanol methanol, diesel etc.) For instance, products from the DEMTROL product range from DOW, the Tretolite product range of Baker Hughes, the Anti-foam Maxamine 70 B of GE Betz; or the de-emulsifier Maxamine 82B van GE Betz or products from the Witbreak range from AKZO may be used.

In a preferred embodiment, one or more chemical additives for separating oil/water emulsions into oil and water selected from de-emulsifiers are added to the streams entering the oil/water separator or to the emulsions in the oil/water separator. The amount of de-emulsifier is suitably equal to or less than 1 vol % of the total liquid stream going into the separator, preferably equal to or less than 0.1 vol %, more preferably equal to or less than 0.01 vol %, the amount preferably being equal to or more than 1 ppmv (parts per million by volume), more preferably 20 ppmv of the total liquid stream going into the separator.

The one or more de-emulsifiers may be added into the process streams under a wide range of temperature, pressure and phase conditions. The one or more de-emulsifiers may be available in both aqueous and hydrocarbon phases.

The above mentioned dry gas stream (i.e. a gas comprising hydrogen, methane, ethane, ethene and optionally nitrogen) and the so-called LPG stream (i.e. a stream comprising C3-C4 hydrocarbon compounds such as propane, propene, butane and butane) may each contain a certain amount of sulphur, for example in the form of hydrogen sulphide or mercaptans. Such a dry gas stream and/or LPG stream, respectively such a C1-C2 compound fraction and/or such a C3-C4 compound fraction are therefore preferably forwarded to an amine treating process to reduce the content of hydrogen sulphide and/or CO2. Mercaptans may suitably be removed by means of a caustic wash.

Amine gas treating, also known as gas sweetening or acid gas removal, refers to a process in which an aqueous solution of one or more alkylamines is used to remove hydrogen sulphide from a gas stream. In addition also carbon dioxide can be removed. Preferred alkylamines are monoethanolamine (MEA), diethanolamine (DEA), methyldiethanolamine (MDEA), diisopropanolamine (DIPA) and diglycolamine (DGA). Optionally also a physical solvent, e.g. sulfolan, may be present. The main equipment pieces in the amine treater are an absorber and a regenerator. In the absorber a downflowing amine solution can absorb hydrogen sulphide and optionally carbon dioxide from an upflowing sour gas stream to produce a sweetened gas stream (no hydrogen sulphide/carbon dioxide) and an amine solution rich in the absorbed sour gasses (also referred to as rich amine solution or rich amine). The resulting rich amine can then be introduced in the top of a regenerator (a stripper with a reboiler) to produce a stripped overhead gas and regenerated or lean amine solution, which regenerated or lean amine solution is recycled to the absorber. Each absorber in an amine treater preferably has its own regenerator, but is also possible to use a common regenerator for a number of absorbers.

Application of some embodiments of the invention may suitably mitigate the formation of so-called sour water emulsions when catalytically cracking a bio-feed in an FCC unit. This may conveniently avoid or solve any waste water treatment plant operation problems (i.e. there may be less organic waste and/or dissolved hydrocarbon compounds slipping to the waste water plant), it may enable the refinery to meet the quality specifications of the FCC products (better sulphur/CO2 removal and possibly reducing chemical oxygen demand), and it may reduce fresh amine replacement cost. The downstream FCC processes (i.e. the product work-up processes) may operate more stable and more efficiently than without the use of de-emulsifiers according to some embodiments of the invention.

By breaking the emulsions in the oil/water separators, the sour water may not carry excess hydrocarbons to the downstream waste water treatment plant. Excessive hydrocarbon carry by the sour water can upset the waste water treatment plant and result in unstable plant operation and higher sulfur oxide air emissions. Also the upset may result in increased chemical and biological oxygen demand (COD, BOD) which may threaten non-compliance of water discharge quality requirements.

Therefore, embodiments of the present invention are well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, substituted, or modified and all such variations are considered within the scope and spirit of the present invention. The invention illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount whether accompanied by the term “about” or not. In particular, the phrase “from about a to about b” is equivalent to the phrase “from approximately a to b,” or a similar form thereof. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces. If there is any conflict in the usages of a word or term in this specification and one or more patent or other documents that may be incorporated herein by reference, the definitions that are consistent with this specification should be adopted.

1. A process for the fluid catalytic cracking of oxygenated hydrocarbon compounds from biological origin, the process comprising:
a) contacting a feed comprising the oxygenated hydrocarbon compounds from biological origin with a fluid cracking catalyst at a temperature of equal to or more than 400° C. to produce a products stream;
b) separating fluid cracking catalyst from the products stream and separating a fraction comprising one or more C1-C4 hydrocarbon compounds from the products stream; and
c) processing the fraction comprising one or more C1-C4 hydrocarbon compounds in a work-up process, which work-up process comprises one or more oil/water separation steps;
wherein one or more de-emulsifiers are added to one or more oil/water separation steps.

2. The process of claim 1, wherein the one or more oil/water separation steps are carried out in one or more separators chosen from the group consisting of a main fractionator overhead separator, a wet gas compressor discharge separator, one or more high pressure separator(s) and/or a butanizer overhead separator.

3. The process of claim 1, wherein the fraction comprising C1-C4 compounds is cooled to obtain a cooled gas stream and a liquid oil/water condensate, followed by separation of the oil and the water fraction in an oil/water separation step.

4. The process of claim 3, wherein the cooled gas stream, before the further separation, is compressed to a pressure between 0.5 and 5 MegaPascal, where after the compressed gas stream is cooled to obtain a cooled compressed gas stream and a liquid oil/water condensate, followed by separation of the oil and the water fraction in an oil/water separation step.

5. The process of claim 1, wherein a fraction comprising C3-C4 compounds is obtained, which fraction is cooled to obtain a cooled gas stream and a liquid oil/water condensate, followed by separation of the oil and the water fraction in an oil/water separation step.

6. The process of claim 1, wherein step c) comprises
cooling at least part of the fraction comprising C1-C4 compounds to form a cooled gas/liquid mixture and separating the cooled gas/liquid mixture in a main fractionator overhead separator into a cooled gas stream, an oil phase and a water phase.

7. The process of claim 6 further comprising:
compressing at least part of the cooled gas stream to form a compressed gas/liquid mixture and separating the compressed gas/liquid mixture in a wet gas compressor discharge separator into a compressed gas stream, an oil phase and a water phase.

8. The process of claim 7 further comprising:
washing the compressed gas stream one or more times with water and/or steam to form one or more washed gas/liquid mixture(s) and separating such one or more washed gas/liquid mixture(s) in one or more high pressure separator(s) into a washed compressed gas stream, an oil phase and a water phase.

9. The process of claim 7 further comprising:
separating the compressed gas stream into a dry gas stream comprising C1-C2 hydrocarbon compounds and a LPG stream comprising C3-C4 hydrocarbon compounds;
cooling the LPG stream comprising C3-C4 hydrocarbon compounds to form a cooled LPG gas/liquid mixture; and
separating the cooled LPG gas/liquid mixture in a butanizer overhead separator into an LPG gas stream, an oil phase and a water phase;
wherein one or more de-emulsifiers are added to one or more of the separators.

10. The process of claim 1, wherein the fraction comprising C1-C4 compounds comprises products of catalytically cracking of a tri-glycerides and/or catalytically cracking of one or more free fatty acids.

11. The process of claim 1, wherein the one or more de-emulsifiers are added to the streams entering a oil/water separator or to the emulsions in a oil/water separator.

12. The process of claim 1, wherein the one or more de-emulsifiers are chosen from the group consisting of (alkyl)phenol-formaldehyde resins, epoxy resins, amines, polyamines, amides, di-epoxides, alcohols, polyols, polyol block copolymers, and the alkoxylated, especially ethoxylated or propoxylated, derivatives there from.

13. The process of claim 1, wherein the one or more de-emulsifiers are added in an amount of equal to or less than 0.1 vol %, and equal to or more than 1 ppmv (parts per million by volume) of the total liquid stream going into a separator.
